(19) United States
(12) Patent Application Publication
(10) Pub. No.: US 2007/0260310 A1
(43) Pub. Date: Nov. 8, 2007

(54) Accommodative Intraocular Lens Having Defined Axial Compression Characteristics

(76) Inventor: Gary A. Richardson, Rochester, NY (US)

Correspondence Address: Bausch & Lomb Incorporated, One Bausch & Lomb Place, Rochester, NY, 14604-2701, US

(21) Appl. No.: 11/745,603

(22) Filed: May 8, 2007

Related U.S. Application Data
(60) Provisional application No. 60/798,548, filed on May 8, 2006.

(51) Int. Cl. A61F 2/16

(52) U.S. Cl. 623/6.34; 623/6.37

(57) ABSTRACT

A multi-optic accommodating intraocular lens (A-IOL) for implantation in a capsular bag of an eye having an optical axis, includes a posterior component, an anterior component that is translatable relative to the posterior component along an optical axis of the A-IOL, and a biasing element that joins at least a portion of the anterior component and at least a portion of the posterior component. The A-IOL is quantitatively characterized by an axial compression characteristic such as a spring constant or an axial restoring force. The axial compression characteristic is capable of keeping the components sufficiently vaulted apart for enabling near vision yet weak enough to allow the eye's accommodative mechanism to pull the optics close together for distance vision.

### CROSS-REFERENCE

This application claims the benefit of Provisional Patent Application No. 60/798,548 filed May 8, 2006, which is incorporated by reference herein.

### FIELD OF THE INVENTION

Embodiments of the invention are generally directed to the field of accommodating intraocular lenses (A-IOLs) and more particularly to a multi-component A-IOL having defined axial compression characteristics, and to a method for providing an A-IOL having such characteristics.

### BACKGROUND OF THE INVENTION

FIG. 1 shows a cross sectional view of the anterior segment of the human eye 20. Reference numerals 22, 26 and 28, respectively, identify the cornea, the iris and the anterior chamber. The natural crystalline lens 32 is situated within an elastic membrane 34 called the capsular bag or lens capsule. The capsular bag 34 is surrounded by and suspended within the ciliary body or muscle 30 by ligament-like structures called zonules 36.

To facilitate vision, the cornea 22 and the lens 32 cooperate to focus incoming light to form an image on the retina (not shown) at the rear of the eye. In the process known as accommodation, the shape of the lens 32 is altered (and its refractive properties thereby adjusted) to allow the eye 20 to focus on objects at varying distances. A typical healthy eye has sufficient accommodation to enable focused vision of objects ranging in distance from infinity (generally defined as over 20 feet from the eye) to very near (closer than 10 inches). When the ciliary muscle 30 is in a relaxed condition, the tension in the zonules 36 increases to exert an equatorial stretching force on the capsular bag 34. Because a healthy crystalline lens 32 has a natural elasticity, this stretching force causes the lens to take on a more flattened, thinner shape as measured along the optical axis 23. Thus when the ciliary muscle is relaxed, the natural lens is in an unaccommodated state suited for distance vision. Accommodation occurs when the ciliary muscle tenses and contracts, which decreases the tension in the zonules 36, allowing the lens to assume a fatter or shorter shape that in cross-section resembles that of a football.

In response to various physiological conditions, the most notable being the occurrence of cataracts, the natural crystalline lens may be removed and replaced by an intraocular lens (IOL). The implantation of an accommodating IOL (A-IOL) is intended to re-establish the accommodative ability (to a lesser or greater degree) of the eye and eliminate the need for additional lenses for focusing near-vision objects. A-IOLs may be of the single optic or multi-component (e.g., two-optic) type. A two-optic A-IOL will generally provide more focusing power and accommodative range than a single-optic A-IOL.

The accommodative operation of a two-optic A-IOL is similar to that described above for the natural crystalline lens. With reference to FIG. 2, an exemplary A-IOL 40 has replaced the natural lens 32 (FIG. 1) and thus is fitted within the evacuated capsular bag 34. The capsular bag, however, is no longer continuously intact; rather, a hole or rhexis 41 has been made in the anterior central region of the capsular bag for removal of the cataracteous tissue and implant of the A-IOL 40. Generally speaking, the A-IOL will consist of an anterior optic component 42, a posterior optic component 44 and a flexible biasing element 46 that connects the two components and allows for their relative axial translation. The biasing element maintains the exemplary A-IOL 40 in a condition of maximum optic separation distance similar to the fattened, accommodating state of the natural crystalline lens. When the ciliary muscle relaxes, the zonules 36 tense and pull radially on the capsular bag 34. This results in what will be referred to herein as a ‘pinching force’ (−)F, exerted in a substantially axial direction as shown in FIG. 2. In response to (−)F, the biasing element 46 of A-IOL 40 flexes or deforms in some manner allowing the anterior optic 42 to move toward the posterior optic 44 along the optical axis 23. When the optics have achieved a desired minimum optic separation distance, the A-IOL 40 will be in an unaccommodated state for distance vision.

The structural configuration of an exemplary A-IOL 40 is illustrated in FIG. 3. The structural components of the A-IOL are disclosed, for example, in U.S. Pat. Nos. 5,275,623; 6,423,094; 6,488,708; and U.S. Published Application Nos. 2004/0015236 and 2003/0130732, the disclosures of which are herein incorporated by reference in their entireties to the fullest extent allowed by applicable laws and rules. A variation of this design is set forth in Applicant's copending application entitled ACCOMMODATIVE INTRAOCULAR LENS WITH COMPRESSIBLE BIASING ELEMENT, filed on this same date as the instant application, the disclosures of which is herein incorporated by reference in its entirety. Other A-IOL designs incorporating proprietary biasing element structures or their equivalents are known in the art. They are described, for example, in U.S. Pat. Nos. 6,695,881 and 6,858,040, the disclosures of which are fully incorporated herein by reference to the fullest extent allowed by applicable laws and rules. As is known in the art, A-IOLs may be manufactured from different materials including, but not limited to, various silicone formulations, polymethylmethacrylate (PMMA) or other suitable materials selected to provide visual clarity, refractive capability, biocompatibility and mechanical stability. Regardless of the mechanical design and material composition, the A-IOL must have a spring constant (i.e., a resistance to an axial compression force) that is capable of keeping the optics sufficiently vaulted apart for enabling near vision yet weak enough to allow the eye's accommodative mechanism to pull the optics close together for distance vision. Additionally, consideration must be given to A-IOL rigidity so that the lens can maintain its own shape in the capsular bag, as well as to lateral stability to maintain alignment between the front and rear optics. These issues are all the more problematic because of the difficulty in determining the maximum force exerted by the ciliary process. In view of the foregoing, the inventors have recognized a need for an A-IOL having practical and well-defined axial compression force characteristics. Embodiments of the invention described herein below will address such needs and illustrate the benefits associated therewith.

### SUMMARY OF THE INVENTION

An embodiment of the invention is directed to a multi-component accommodating intraocular lens (A-IOL) that has a quantitatively defined axial compression characteristic. In an exemplary aspect, the A-IOL is characterized by the spring equation of Hooke's Law, F=−kx, where k is defined as the spring constant, x is the extension (displacement) of the spring and F is the axial compression force exerted by the spring in direct opposition to the direction of displacement. The A-IOL includes a posterior component, an anterior component that is translatable relative to the posterior component along an optical axis of the A-IOL and a biasing element that connects to at least a portion of the anterior component and at least to a portion of the posterior component. In an aspect, the A-IOL has an axially directed spring constant between about 0.9 to 2.50 milli-Newtons per millimeter (mN/mm), and in some embodiments, preferably between 1.0 to 1.6 mN/mm. In some embodiments, according to the above aspect, the A-IOL is characterized by having a variable component separation distance, X, where 0.1<X<1.9 millimeters (mm). In some embodiments according to the above aspect, the A-IOL is characterized by a restoring force (i.e., a resistance to an axial compression) of between about 0.25 to 2.45 milli-Newtons (mN) when X is varied between 1.9 and 0.1 mm.

In a typical aspect according to the embodiment, both the posterior component and the anterior component have optical power. In an alternative aspect, the posterior component will include a frame having an aperture with no optical power.

The biasing element may be of integrated or piece-wise construction. It may be continuous or include distinct anterior and posterior portions, regions, segments, etc. The A-IOL may include a plurality of biasing elements spaced about the anterior and posterior components. According to an aspect, one or more of the biasing elements may have a spring constant modifying feature that acts as a static control to modify the spring constant of the A-IOL.

The biasing elements, as well as the A-IOLs themselves, can be manufactured by known techniques including, but not limited to, molding, casting and laser trimming. The materials used for the A-IOL and its component structures, whether of completely unitary construction or multi-element construction, comprise known materials for manufacturing A-IOLs including, but not limited to, silicone formulations, polymethylmethacrylate (PMMA) or other suitable materials that provide visual clarity, refractive capability, biocompatibility and mechanical stability. The anterior optic and the posterior component of A-IOLs according to the embodiments of the invention may have any suitable optical characteristics. As such, lens power distribution, lens shapes, translation ranges and other parameters can be selected to suit patient and manufacturing requirements.

Another embodiment of the invention is directed to a method for designing a multi-component accommodating intraocular lens (A-IOL) having a defined axial compression characteristic. This method involves the steps of selecting an A-IOL design that includes an anterior component, a posterior component and a biasing element connected to at least a portion of the anterior component and to at least a portion of the posterior component, determining a suitable A-IOL optical power range, accommodative range and component separation distance between an accommodating state and a non-accommodating state of the A-IOL, and determining a structural configuration of the A-IOL and/or a suitable biasing element material having an elastic modulus and shape that provides the A-IOL with a spring constant that is sufficient to keep the anterior and posterior components sufficiently vaulted apart for a near vision state of the A-IOL and to allow a desired translational compression of the components for enabling a distance vision state of the A-IOL in response to a force exerted by a ciliary process of a human eye.

Another embodiment of the invention is directed to method for modifying an axial compression characteristic of a multi-component accommodating intraocular lens (A-IOL). The method involves the steps of providing an A-IOL that includes an anterior component, a posterior component and a biasing element connected to at least a portion of the anterior component and to at least a portion of the posterior component and providing a spring constant modifying feature in the biasing element to statically modify an axially directed spring constant value of the A-IOL.

The various benefits and advantages of the A-IOL embodiments of the invention will be evident to a person skilled in the art in view of the drawing figures and the following detailed description, and as defined in the appended claims.

### BRIEF DESCRIPTION OF THE DRAWINGS

FIG. 1 is a cross sectional view of the anterior portion of a human eye containing a natural crystalline lens;

FIG. 2 is a cross sectional view of the anterior portion of a human eye containing an exemplary A-IOL according to an embodiment of the invention;

FIG. 3 is a schematic perspective view of an A-IOL according to an exemplary embodiment of the invention;

FIG. 4 is a cross sectional schematic view of an illustrative A-IOL showing the axial compression force parameters according to an embodiment of the invention;

FIG. 5 is a partial cross sectional schematic view of an A-IOL according to an exemplary embodiment of the invention;

FIG. 6 is a three-dimensional iso-view of an A-IOL according to an exemplary embodiment of the invention in a natural, uncompressed state and in a compressed state;

FIGS. 7A, 7B, 7C, respectively, are photo reproductions of three aspects of an A-IOL according to an exemplary embodiment of the invention;

FIG. 8 is a graph of axial compression force versus optic compression distance for various aspects of exemplary A-IOLs according to an embodiment of the invention;

FIG. 9 is a pictorial illustration of a system for generating the data in the graph of FIG. 8;

FIG. 10 is a schematic illustration providing a perspective view of an embodiment of a lens according to aspects of the present invention in which the anterior lens element is recessed relative to the haptics; and

FIG. 11 is a cross-section side view illustrating further details of the recessed anterior optic of the AIOL in FIG. 10.

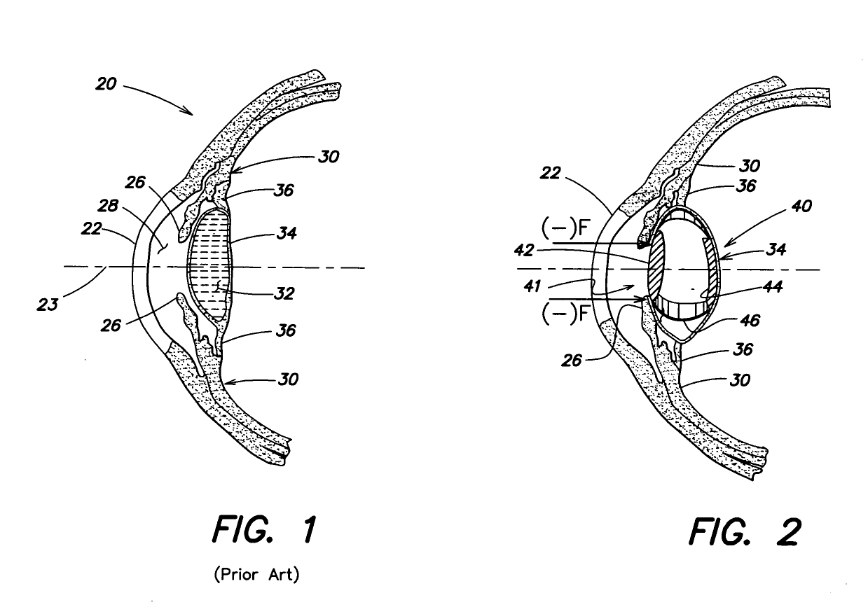

### DETAILED DESCRIPTION OF AN EMBODIMENT OF THE INVENTION

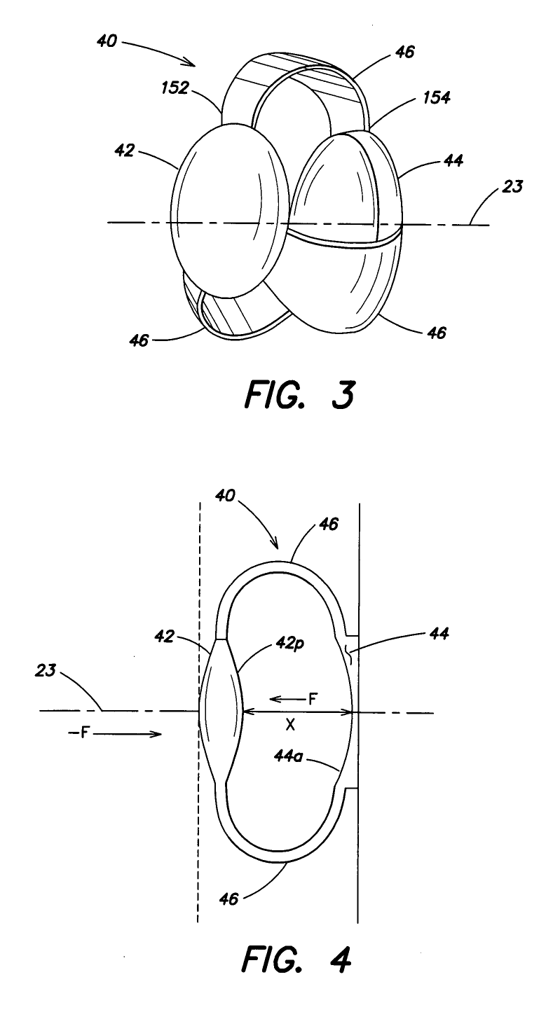

An exemplary A-IOL 40 for implantation into the capsular bag of an eye in place of the natural crystalline lens is shown in FIG. 3. A-IOL 40 is configured to change the refractive properties of the eye in response to the eye's natural process of accommodation as described above. The A-IOL 40 includes an anterior optic component 42, a posterior optic component 44 and three identical, single-piece, flexible biasing elements 46, which join the anterior and posterior optics and facilitate the relative translational movement of the components along the optical axis 23. (It will be understood that when the A-IOL is implanted, the posterior optic is intended to remain stationary in the posterior region of the capsular bag, while the anterior optic translates along the optical axis for accommodative effect). In alternative aspects, two biasing elements or four or more biasing elements may be provided. In another alternative aspect, the biasing element 46 may be of one piece and joined continuously about the peripheral edges of the anterior and posterior optics. In some embodiments, biasing elements 46 are equally spaced about the peripheries of the anterior and posterior components. Each biasing element has an anterior end region 152 that joins at least a portion of the anterior optic 42 and a posterior end region 154 that joins at least a portion of the periphery of the posterior optic 44. Based upon material selection criteria of the biasing element 46 including elastic modulus, structural parameters of the A-IOL including biasing element number, size, shape and thickness, mechanical requisites of the A-IOL such as minimum and maximum optic separation, optical considerations such as optical power, accommodative range and other criteria recognized by a person skilled in the art, the A-IOL 40 will have an axial compression characteristic, for example, a spring constant, k, where 0.09<k<1.50 milli-Newton per millimeter (mN/mm) of variable component separation distance, X.

FIG. 4 illustrates the relationship of the parameters: force, F, axial displacement, X, and spring constant, k as used in the spring equation form of Hooke's Law, F=−kX. X represents the variable distance between the posterior surface 42p of anterior optic 42 and the anterior surface 44a of posterior optic 44 as anterior optic 42 translates toward (−X) posterior optic 44 along optical axis 23 in response to a force, (−)F, referred to above as a pinching force resulting from relaxation of the ciliary muscle of the eye during the accommodation process. In its unstressed state, the biasing elements 46 exert an opposite (restoring) axial force, F, to separate the components of the A-IOL 40 to their designed maximum separation distance (+)X. As a result of experimentation and empirical evidence, the inventors have determined the appropriate value range for the spring constant, k, recited above. In some embodiments k is in the range 0.9 to 2.50 milli-Newtons per millimeter (mN/mm), and in some embodiments, preferably between 1.0 to 1.6 mN/mm. These value ranges are not to be construed as limited to the particular design of exemplary A-IOL 40; rather, they are applicable to various alternative designs, for example, those designs disclosed in the publications referred to in the Background section of the instant description.

According to an illustrative aspect, the component separation distance, X, can be in the range of zero to approximately 3mm. To avoid potential sticking problems when the components touch in the fully compressed state, the A-IOL may be designed to have a minimum component separation distance that is greater than zero. An exemplary minimum separation value is 1 mm, however, this distance can be a different value depending upon optical and mechanical characteristics of the particular A-IOL. In a particular aspect, X<1.9 mm. In a more particular aspect, 0.1<X<1.9 mm. Exemplary optical parameters for these ranges can include an anterior component optical power of about 36 diopters and a posterior component optical power in the range between about −25 to zero diopters and more particularly between about −20 and −10 diopters. An exemplary accommodative range for A-IOL 40 is 4 diopters.

According to an exemplary embodiment of the invention, an A-IOL 40 as illustrated in FIG. 3 is characterized as exerting an axial restoring force, F, between the range of about 0.25 to 2.45 mN. In a particular aspect, 0.98<F<2.00 mN. In a more particular aspect, 0.9<k<2.50 mN/mm of component separation distance for the X values described above. More particularly, 1.0<k<1.60 mN/mm for the recited X value ranges.

In regard to all of these values, the underlying principle is that the A-IOL must have a axial restoring characteristic (e.g., spring constant giving rise to a restoring force) that is capable of keeping the optics sufficiently vaulted apart for enabling near vision yet weak enough to allow the eye's accommodative mechanism to pull the optics close together for distance vision. At the same time, consideration must be given to A-IOL rigidity so that the lens can maintain its own shape in the capsular bag, as well as to its lateral stability to maintain alignment between the front and rear optics.

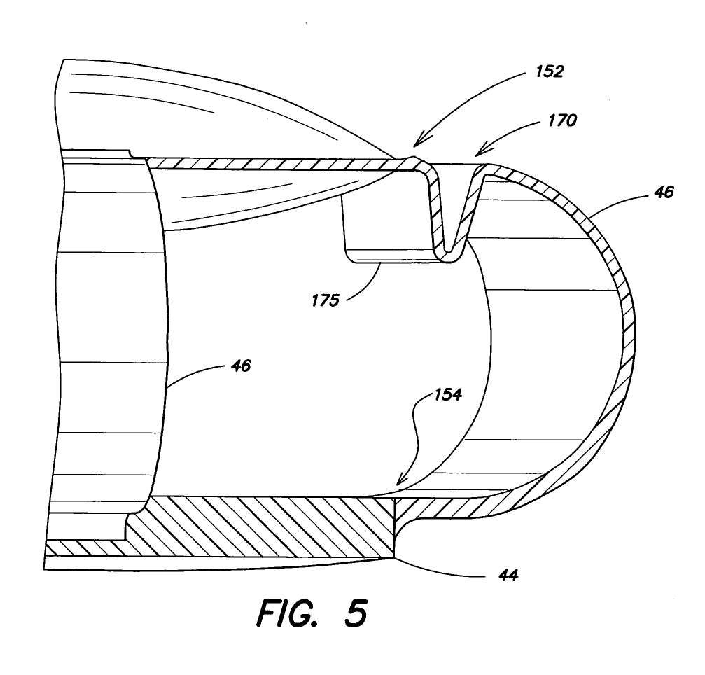

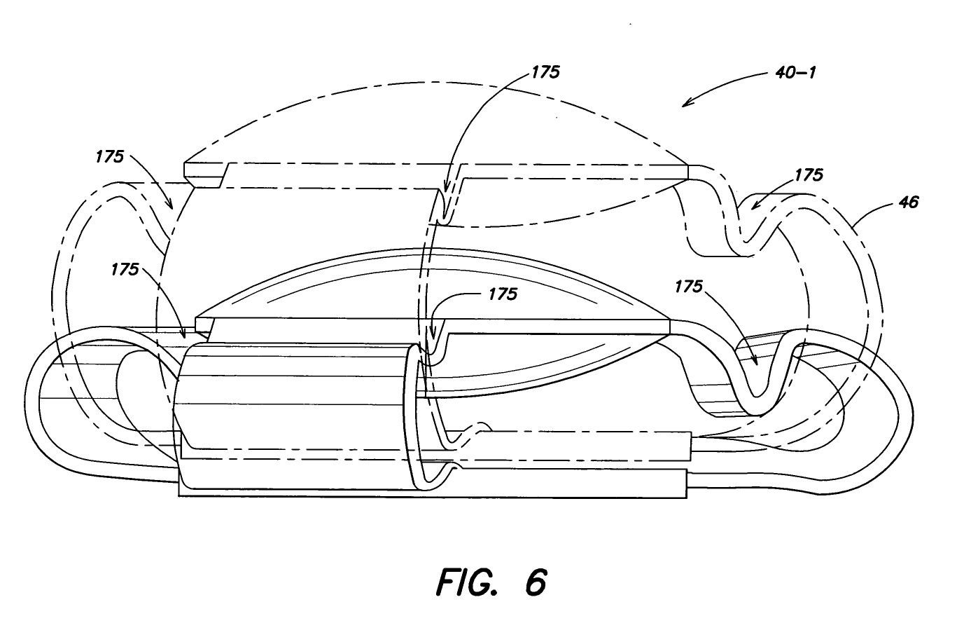

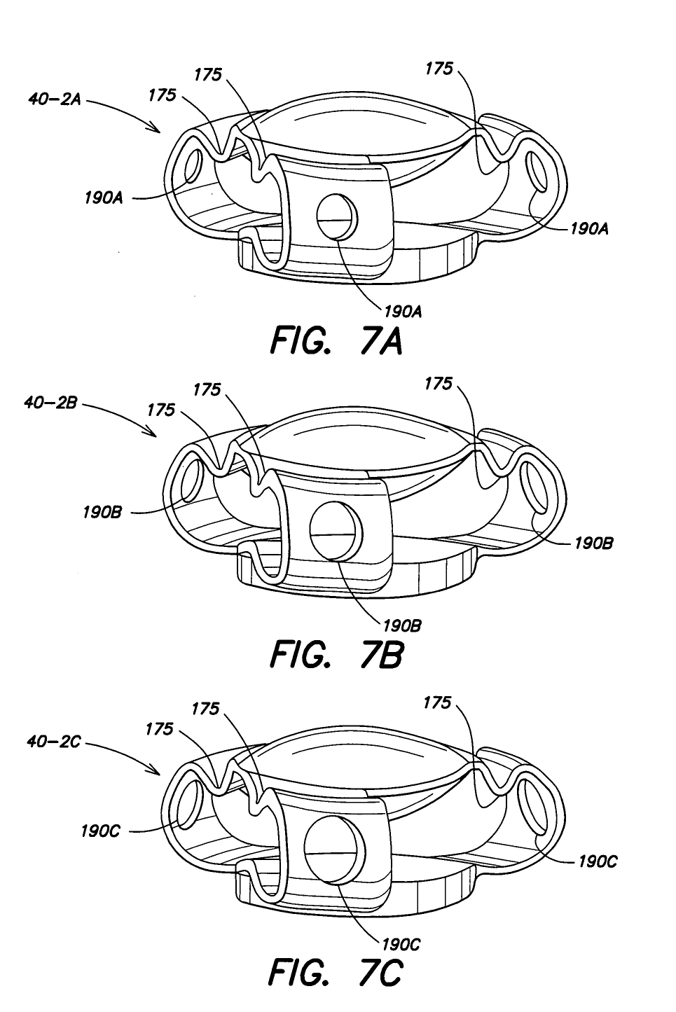

FIG. 5 illustrates another exemplary A-IOL 40-1 according to an embodiment of the invention. The biasing element 46 of A-IOL 40-1 includes a lens diameter modifier feature 170 in the form of a semi-continuous, resiliently deformable gap structure 175, referred to hereinafter as deformation feature 175. Specific details and alternative aspects of the deformation feature 175 are disclosed in applicant's copending patent application entitled ACCOMMODATIVE INTRAOCULAR LENS WITH COMPRESSIBLE BIASING ELEMENT referred to herein above. As illustrated in FIG. 5, the deformation feature 175 is located adjacent the anterior end portion 152 of the respective biasing element 46. Although the deformation feature as disclosed in the referenced application functions to change the diameter of the A-IOL in response to capsular bag shrinkage, it further functions as a static control to modify the spring constant of the A-IOL 40-1 according to the instant embodiment. In an illustrative aspect, the deformation feature has an undeformed gap dimension, GU, the range between 500 to 1000 microns (μ). In an exemplary aspect, the deformation feature 175 is in the form of a semi-continuous, V-shaped gap. Other gap shapes may include, for example, U-shaped, W-shaped, M-shaped, N-shaped, S-shaped, etc The deformation feature 175 may alternatively be located adjacent the posterior end region 154 of the biasing element, at both the anterior and posterior end regions of the biasing element or at another location, e.g., central region, within the biasing element. In an exemplary aspect illustrated, in part, in FIGS. 7A-C, each biasing element 46 has a deformation feature 175 in an anterior end region thereof. FIG. 6 shows a view of A-IOL 40-1 including deformation features 175 in an axially compressed, non-accommodative state and, in phantom, in a natural, accommodating state.

Referring again to FIGS. 7A, 7B and 7C, there are shown three exemplary A-IOLs 40-2A, 40-2B and 40-2C, respectively. In addition to deformation features 175, the biasing elements 46 of the illustrated A-IOLs include alternative forms of a lens diameter modifier feature 190 in the form of circular apertures 190A, 190B and 190C. As illustrated in FIG. 7A, each biasing element 46 has a circular aperture 190A located in a central portion of the biasing element. The aperture has a diameter of about 1 mm. FIGS. 7B, 7C illustrate similar apertures 190B, 190C, respectively, having respective diameters of approximately 1.5 mm and 2 mm. The A-IOLs 40-2A, 40-2B and 40-2C are shown also including deformation features 175, however, embodiments of the invention do not require that both lens diameter modifier features 175 and 190 be present. Furthermore, the apertures 190 need not be circular; rather, they can be of any variety of selected shapes and sizes (e.g., as measured by a major diameter of the aperture), which suitable modify the axial compression characteristic of the A-IOL. In an exemplary aspect, the major diameter of the apertures 190 are in the range between about 1 to 2 mm.

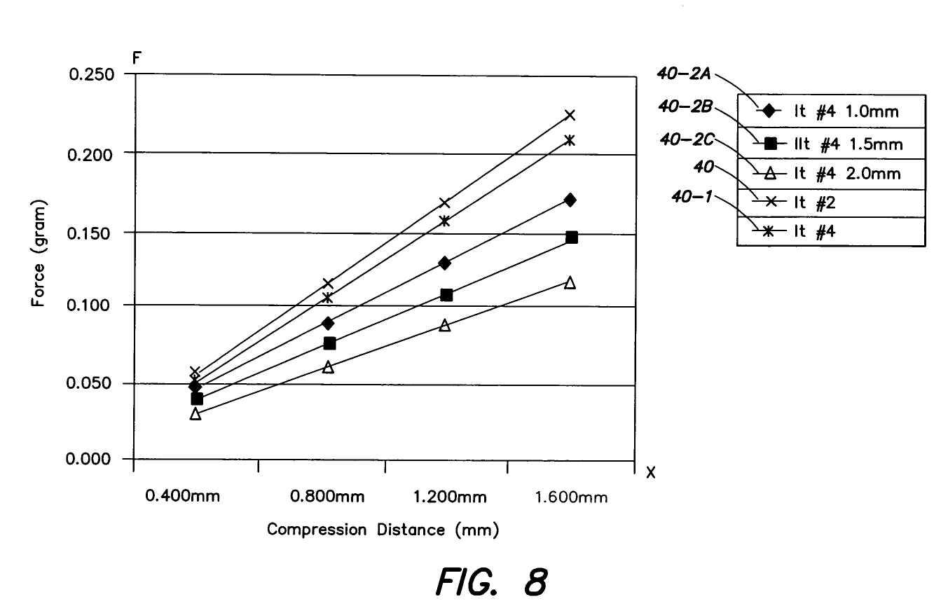

FIG. 8 is a graph of five plots, a, b, c, d, e, of measured axial compression force versus optic compression distance for A-IOLs 40, 40-1, 40-2A, 40-2B and 40-2C, respectively. The slope of each plot represents the spring constant, k, of the exemplary A-IOLs. FIG. 8 quantitatively illustrates the effect of the exemplary lens diameter modifier features of the biasing elements. Although force is expressed in units of grams, the conversion to milli-Newtons (mN) is easily realized, as 1 g equals 9.8 mN. Each curve is expected to remain linear with a constant slope value for a compression distance range, X, between about 0.4 to 2.0 mm to include the A-IOL non-accommodating state and the A-IOL accommodating state.

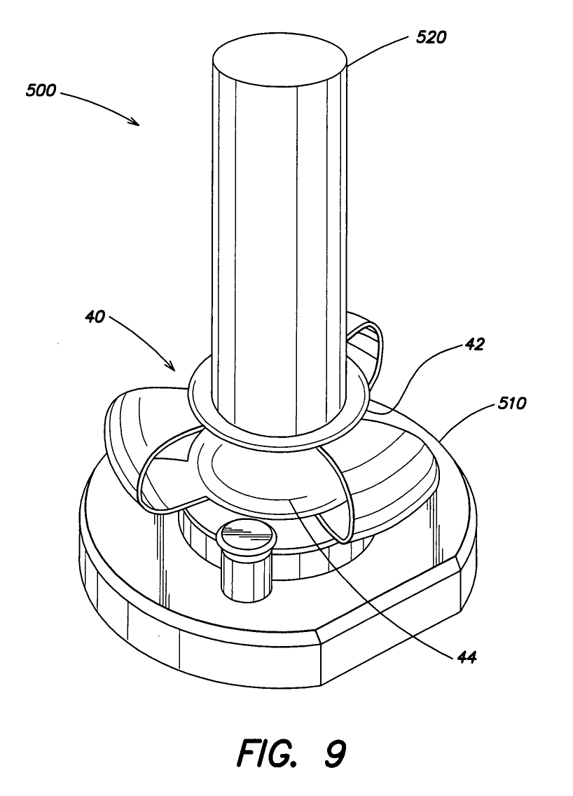

The data for the plots (a-e) of FIG. 8 were generated with the use of a system 500 as pictorially illustrated in FIG. 9 for measurement of exemplary A-IOL 40. The system 500 was composed of a lens holding fixture 510 that holds the posterior component 44 of the A-IOL 40. A compression probe 520 was integrated to a force transducer (not shown) accurate to 0.001 grams, and to a linear displacement gage (to measure the axial component displacement) accurate to 0.01 mm. Procedurally, the A-IOL was mounted in the lens holding fixture. The probe was then lowered until it contacted the surface of the anterior optic 42 and compressed the optic toward the posterior component 44 to a predefined accommodative state, or until the optical component surfaces touched. The axial displacement of the anterior lens was then measured. The measured axial displacement was represented with the variable, x. Then, the amount of axial force that required to compress the anterior lens the distance, x, was measured and represented with the variable, F. Values of F versus x were plotted in FIG. 8.

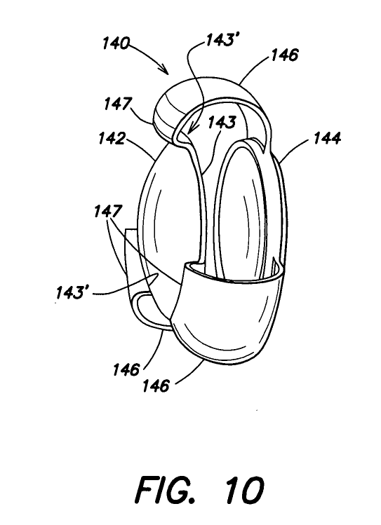

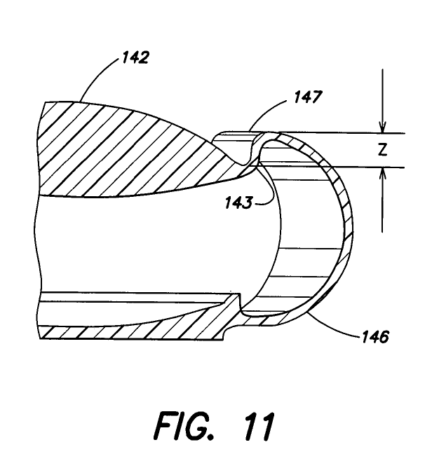

FIG. 10 is a schematic illustration providing a perspective view of an embodiment of a lens according to aspects of the present invention in which the anterior lens element is recessed relative to the haptics. AIOL 140 comprises an anterior optical element 142; a posterior component 144; and three longitudinal haptics 146 each coupled to at least a portion of the anterior optic and at least a portion of the posterior component. AIOL 140 substantially conforms to the interior surface of the capsular bag 34 (shown in FIG. 2), other than at the anterior optic. As is apparent in FIG. 11, each haptic 146 has an anterior-most portion 147 that is disposed more anteriorly than an edge 143 of the anterior optic. In particular, the edge is located in the portion 143′ where the haptic couples to the anterior lens. As illustrated in FIG. 11, the anterior-most potion 147 of haptic 146 is disposed approximately 0.5≦Z≦0.8 mm more anteriorly located than edge 143 of the anterior optic. In some embodiments, the anterior-most potion 147 of haptic 146 is disposed approximately 0.6 mm more anteriorly located than edge 143 of the anterior optic. Further details are given in an application titled ACCOMMODATING INTRACULAR LENS HAVING A RECESSED ANTERIOR OPTIC, attorney docket no. P04056, filed on even date herewith. The substance of said application is hereby incorporated by reference.

The above disclosed embodiments of an A-IOL support associated method embodiments of the invention. A method for designing a multi-component accommodating intraocular lens (A-IOL) having a defined axial compression characteristic involves the steps of selecting an A-IOL design that includes an anterior component, a posterior component and a biasing element in operable connection to at least a portion of the anterior component and to at least a portion of the posterior component. A suitable A-IOL optical power range, accommodative range and component separation distance between an accommodating state and a non-accommodating state of the A-IOL are determined. A structural configuration of the A-IOL and/or a suitable biasing element material is also determined. A suitable biasing element material should have an elastic modulus that provides the A-IOL with a spring constant that is sufficient to keep the anterior and posterior components sufficiently vaulted apart for a near vision state of the A-IOL and to allow a desired translational compression of the components for enabling a distance vision state of the A-IOL in response to a force exerted by a ciliary process of a human eye. As described above, an axial compression characteristics of the A-IOL such as its spring constant, for example, can be modified by incorporating structural feature modifications to the biasing element of the A-IOL. Exemplary features include gap structures and apertures of suitable shape and size.

In an exemplary design, the optical power capability of the A-IOL is about 20 diopters. An exemplary optical power range is between about 10 to 30 diopters. An exemplary accommodative range is about four diopters over a selected optic separation distance of between about 0.4 to 2.0 mm. An exemplary A-IOL is designed to have a spring constant, k, where 0.09<k<1.50 mN/mm of variable component separation distance.

The foregoing description of the embodiments of the invention have been presented for the purposes of illustration and description. It is not intended to be exhaustive or to limit the invention to the precise form disclosed. Many modifications and variations are possible in light of the above teaching. It is intended that the scope of the invention be limited not by this detailed description, but rather by the claims appended hereto.

We claim:

1. A multi-component accommodating intraocular lens (A-IOL), comprising
a posterior component;
an anterior component that is translatable relative to the posterior component along an optical axis of the A-IOL; and
a biasing element that joins at least a portion of the anterior component and at least a portion of the posterior component,
wherein the lens is characterized by having an axially directed spring constant, k,
where 0.9<k<2.50 milli-Newton per millimeter (mN/mm).

2. The A-IOL of claim 1, where 1.0<k<1.6 mN/mm.

3. The A-IOL of claim 1, wherein the spring constant occurs over a component separation
distance X, where X≦1.9 millimeters (mm).

4. The A-IOL of claim 3, wherein the spring constant occurs over a component separation distance X, where 0.1≦X≦2.85 mm.

5. The A-IOL of claim 1, wherein the anterior component is recessed relative to the anterior-most portion of the biasing element.

6. The A-IOL of claim 5, wherein the anterior-most potion of the biasing element is disposed approximately 0.5 to 0.8 mm more anteriorly located than edge of the anterior optic.

7. The A-IOL of claim 2, wherein the anterior optic is recessed relative to the anterior-most portion of the biasing element.

8. The A-IOL of claim 7, wherein the anterior-most potion of the biasing element is disposed approximately 0.5 to 0.8 mm more anteriorly located than edge of the anterior optic.

9. The A-IOL of claim 3, wherein the lens is further characterized by an axial compression force, F, where 0.25≦F≦2.45 mN.

10. The A-IOL of claim 9, wherein 0.98≦F≦2.00 mN.

11. The A-IOL of claim 1, wherein each of the posterior component and anterior component is an optic having an optical power.

12. The A-IOL of claim 1, wherein the anterior component is an optic having an optical power and the posterior component has no optical power.

13. The A-IOL of claim 12, wherein the posterior component has an aperture.

14. The A-IOL of claim 1, comprising a plurality of biasing elements.

15. The A-IOL of claim 14, wherein said plurality of biasing elements are equally spaced about the anterior and posterior components.

16. The A-IOL of claim 15, wherein at least one of said plurality of biasing elements has a lens diameter modifier feature.

17. The A-IOL of claim 16, wherein the lens diameter modifier feature is a semi-continuous gap structure.

18. The A-IOL of claim 17, wherein the gap structure is resiliently deformable and has an undeformed gap dimension between about 500 to 1000 microns.

19. The A-IOL of claim 17, wherein the gap structure has a shape in the form of one of a U-shaped gap, a V-shaped gap, a C-shaped gap, a W-shaped gap, an M-shaped gap and an N-shaped gap.

20. The A-IOL of claim 16, wherein the lens diameter modifier feature is an aperture having a selected size and shape.

21. The A-IOL of claim 20, wherein the aperture has a major diameter, d, where d is in the range between about 1-2 mm.

22. The A-IOL of claim 21, wherein d has a value of about 1 mm.

23. The A-IOL of claim 21, wherein d has a value of about 1.5 mm.

24. The A-IOL of claim 21, wherein d has a value of about 2 mm.

25. A multi-component accommodating intraocular lens (A-IOL), comprising
a posterior component;
an anterior component that is translatable relative to the posterior component along an optical axis of the A-IOL; and
a biasing element that joins at least a portion of the anterior component and at least a portion of the posterior component,
wherein the lens is characterized by having an axial compression force, F, where 0.25≦F≦2.45 milli-Newtons (mN).

26. The A-IOL of claim 25, wherein 0.98≦F≦2.00 mN.

27. The A-IOL of claim 25, having a variable component separation distance, X, where X≦1.9 millimeters (mm).

28. The A-IOL of claim 27, where 0.1≦X≦1.9 mm.

29. The A-IOL of claim 25, wherein the anterior optic is recessed relative to the anterior-most portion of the biasing element.

30. The A-IOL of claim 29, wherein the anterior-most potion of the biasing element is disposed more than 0.5 mm more anteriorly located than edge of the anterior optic.

31. The A-IOL of claim 30, wherein the anterior-most potion of the biasing element is disposed approximately 0.5 to 0.8 mm more anteriorly located than edge of the anterior optic.

32. The A-IOL of claim 31, wherein the anterior-most potion of the biasing element is disposed approximately 0.6 mm more anteriorly located than edge of the anterior optic.

33. The A-IOL of claim 25, wherein the A-IOL is a silicone material having an elastic modulus value equal to about 1 Mpa.

34. The A-IOL of claim 25, having a spring constant, k, where 0.9≦k≦2.5 milli-Newton per millimeter (mN/mm) of component separation distance.

35. The A-IOL of claim 34, where 1.0<k≦1.6 mN/mm.

36. The A-IOL of claim 25, wherein each of the posterior component and anterior component is an optic having an optical power.

37. The A-IOL of claim 25, wherein the anterior component is an optic having an optical power and the posterior component has no optical power.

38. The A-IOL of claim 37, wherein the posterior component has an aperture.

39. The A-IOL of claim 25, comprising a plurality of biasing elements.

40. The A-IOL of claim 39, wherein said plurality of biasing elements are equally spaced about the anterior and posterior components.

41. The A-IOL of claim 39, wherein at least one of said plurality of biasing elements has a lens diameter modifier feature.

42. The A-IOL of claim 41, wherein the lens diameter modifier feature is an aperture having a selected size and shape.

43. The A-IOL of claim 42, wherein the aperture has a major diameter, d, where d is in the range between about 1-2 mm.

44. The A-IOL of claim 43, wherein d has a value of about 1 mm.

45. The A-IOL of claim 43, wherein d has a value of about 1.5 mm.

46. The A-IOL of claim 43, wherein d has a value of about 2 mm.

47. The A-IOL of claim 41, wherein the lens diameter modifier feature is a semi-continuous gap structure.

48. The A-IOL of claim 47, wherein the gap structure is resiliently deformable and has an undeformed gap dimension between about 500 to 1000 microns.

49. The A-IOL of claim 47, wherein the gap structure has a shape in the form of one of a U-shaped gap, a V-shaped gap, a C-shaped gap, a W-shaped gap, an M-shaped gap and an N-shaped gap.

50. The A-IOL of claim 25, wherein the A-IOL is of unitary construction.

51. The A-IOL of claim 25, wherein the biasing element has a lens diameter modifier feature.

52. The A-IOL of claim 51, wherein the lens diameter modifier feature is an aperture having a selected size and shape.

53. The A-IOL of claim 52, wherein the aperture has a major diameter, d, where d is in the range between about 1-2 mm.

54. The A-IOL of claim 53, wherein d has a value of about 1 mm.

55. The A-IOL of claim 53, wherein d has a value of about 1.5 mm.

56. The A-IOL of claim 53, wherein d has a value of about 2 mm.

57. A method for designing a multi-component accommodating intraocular lens (A-IOL) having a defined axial compression characteristic, comprising:
selecting an A-IOL design that includes an anterior component, a posterior component and a biasing element in operable connection to at least a portion of the anterior component and to at least a portion of the posterior component;
determining a suitable A-IOL optical power range, accommodative range and component separation distance between an accommodating state and a non-accommodating state of the A-IOL;
determining at least one of a structural configuration of the A-IOL and a suitable biasing element material having an elastic modulus that provides the A-IOL with a spring constant that is sufficient to keep the anterior and posterior components sufficiently vaulted apart for a near vision state of the A-IOL and to allow a desired translational compression of the components for enabling a distance vision state of the A-IOL in response to a force exerted by a ciliary process of a human eye.

58. The method according to claim 57, wherein the suitable A-IOL optical power range is about 20 diopters.

59. The method according to claim 57, wherein the suitable A-IOL optical power range is between about 10 to 30 diopters.

60. The method according to claim 57, wherein the suitable accommodative range is about four diopters.

61. The method according to claim 57, wherein the A-IOL has a spring constant, k, where 0.9≦k≦2.5 milli-Newton per millimeter (mN/mm) of variable component separation distance.

62. The method according to claim 57, wherein the A-IOL has a spring constant, k, where 0.1≦k≦1.6 milli-Newton per millimeter (mN/mm) of variable component separation distance.

63. A method for modifying an axial compression characteristic of a multi-component accommodating intraocular lens (A-IOL), comprising:
providing an A-IOL that includes an anterior component, a posterior component and a biasing element in operable connection to at least a portion of the anterior component and to at least a portion of the posterior component; and
providing a spring constant modifying feature in the biasing element to controllably modify an axially directed spring constant value of the A-IOL.

64. The method according to claim 63, wherein providing the spring constant modifying feature comprises providing a deformation feature in the form of a semi-continuous gap structure.

65. The method according to claim 63, wherein providing the spring constant modifying feature includes providing an aperture of a desired size and shape.

66. The method according to claim 65, comprising providing the biasing element with an aperture having a major diameter of between about 1.0 to 2.0 mm.

67. The method according to claim 66, comprising providing the biasing element with about a 1.0 mm diameter aperture.

68. The method according to claim 66, comprising providing the biasing element with about a 1.5 mm diameter aperture.

69. The method according to claim 66, comprising providing the biasing element with about a 2.0 mm diameter aperture.
